(19) United States
(12) Patent Application Publication
(10) Pub. No.: US 2009/0312641 A1
(43) Pub. Date: Dec. 17, 2009

(54) Method for determining the propagation speed of ultrasonic radiation in a layer of tissue and ultrasound system for performing this method

(75) Inventors: Josef Kozak, Tuttlingen (DE); Henrik Alenkovic, Vilnius (LT)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/454,834

(22) Filed: May 22, 2009

(30) Foreign Application Priority Data
Jun. 17, 2008 (DE) 10 2008 028 736

(51) Int. Cl. A61B 8/15

(52) U.S. Cl. 600/448

(57) ABSTRACT

In a method for determining the propagation speed of ultrasonic radiation in a layer of tissue located in front of a structure reflecting ultrasonic radiation, wherein ultrasonic radiation is transmitted from a transmitter arranged immediately outside the layer of tissue through the layer of tissue onto the structure, and the ultrasonic radiation reflected at the structure is received with a receiver arranged immediately outside the layer of tissue, in order to improve the accuracy of the determination of the speed of propagation, it is proposed that the ultrasonic radiation be transmitted in the form of ultrasonic pulses, and the transit times t₁ and t₂ of the ultrasonic radiation pulses between transmission and reception be determined for at least two different spacings l₁ and l₂ between transmitter and receiver, and the propagation speed of the ultrasonic radiation in the layer of tissue be calculated from the two spacings l₁ and l₂ between transmitter and receiver and from the two transit times t₁ and t₂. An ultrasound system is indicated for performing this method.

The present disclosure relates to the subject matter disclosed in German application number 10 2008 028 736.9 of Jun. 17, 2008, which is incorporated herein by reference in its entirety and for all purposes.

### BACKGROUND OF THE INVENTION

The invention relates to a method for determining the propagation speed of ultrasonic radiation in a layer of tissue located in front of a structure reflecting ultrasonic radiation, wherein ultrasonic radiation is transmitted from a transmitter arranged immediately outside the layer of tissue through the layer of tissue onto the structure, and the ultrasonic radiation reflected at the structure is received with a receiver arranged immediately outside the layer of tissue.

When examining structures in a body, in particular, bone structures, which are covered by a layer of soft tissue, it is necessary to know the speed of propagation of the ultrasonic radiation in the layer of soft tissue. During such an examination, the ultrasonic radiation is directed from a transmitter placed directly on the layer of tissue through the layer of tissue onto the bony structure, reflected by it and then received by a receiver located immediately outside of and adjacent to the layer of tissue. If the speed of propagation of the ultrasonic radiation is known, it is possible to calculate from the time interval between transmission and reception of the ultrasonic radiation the path covered by the ultrasonic radiation from the transmission to the reflection point and then back to the receiver and hence the spacing between the bony structure and the outer side of the layer of tissue. When examining a body, a value is normally assumed for the speed of propagation of the ultrasonic radiation, for example, a speed of 1540 m/s, for this layer of tissue. However, this is a value which deviates from the actual speed of propagation in tissue of a different texture. In fatty tissue, for example, the speed of propagation is 1458 m/s, in muscular tissue 1630 m/s. Therefore, use of the described standard value of 1540 m/s may result in erroneous measurements of the spacing between the outer side of the layer of tissue and the bony structure reflecting the ultrasonic radiation.

An object underlying the invention is to so improve a method of the generic kind that a value can be determined for the speed of propagation of the ultrasonic radiation, which takes better account of the actual propagation speed values for the layer of tissue than the aforementioned standard value.

### SUMMARY OF THE INVENTION

This object is accomplished, in accordance with the invention, in a method of the kind described at the outset in that the ultrasonic radiation is transmitted in the form of ultrasonic pulses, and the transit times t₁ and t₂ of the ultrasonic radiation pulses between transmission and reception are determined for at least two different spacings l₁ and l₂ between transmitter and receiver, and the propagation speed of the ultrasonic radiation in the layer of tissue is calculated from the two spacings l₁ and l₂ between transmitter and receiver and from the two transit times t₁ and t₂.

The different spacings between transmitter and receiver result in different paths for the ultrasonic radiation between transmission, reflection and reception. These paths depend on the spacing between the outer side of the layer of tissue to which transmitter and receiver are applied and the bony structure, and this spacing is initially not known, so that it is not possible to determine the length of the path covered by the ultrasonic radiation. By taking at least two different measurements with different spacings between transmitter and receiver, it is, however, possible to leave this unknown spacing unconsidered and nevertheless on the basis of the transit time measurements along the two different paths to determine an average speed of propagation for the ultrasound in the layer of tissue. This is due to the fact that when calculating the speed of propagation from the transit time measurements, one can proceed on the basis that the spacing between the outer side of the layer of tissue and the bony structure is identical in both measurements and that, in addition, in both cases, the angle of incidence and the angle of reflection of the ultrasonic radiation at the bony structure are identical. Simple equations between the spacings l₁ and l₂ and the two transit times t₁ and t₂ measured in association with these are thereby obtained.

In this determination, it is also assumed that the ultrasound propagation speed in the layer of tissue is identical throughout it. An average speed of propagation is thus determined, which does, however, vary if the layers of tissue are of different composition. The average speed of propagation determined in this way for a layer of tissue does, as a rule, match the respective layer of tissue much better than the standard ultrasound propagation speed discussed at the outset.

It is advantageous for transmitter and receiver to be arranged in a common plane and along a common line for both spacings l₁ and l₂. This line may be a straight line, but it is also possible for it to be a bent line, so that the course of this line matches the outer contour of the layer of tissue.

It is expedient for transmitter and receiver to be so arranged that the centers of their spacing l₁ and l₂, respectively, coincide. This coinciding center can then be arranged immediately above the bony structure to be determined, i.e., directly at the point at which the spacing of the outer surface of the layer of tissue from the bony structure is to be measured.

This method can be performed particularly advantageously when the ultrasonic radiation emanating from the transmitter is focused in the area of the structure. Using suitable techniques, it is possible to not transmit the ultrasonic radiation homogeneously from the transmitter, but to achieve a focusing in a certain direction and at a certain distance from the transmitter, so that the intensity of the ultrasonic radiation is particularly high in this area. This can be achieved using lens-type components of the ultrasound transmitter. In a particularly preferred method, the focusing is brought about by several adjacent ultrasound transmitters being simultaneously activated so that the ultrasonic rays emanating from these ultrasound transmitters are superimposed on one another and as a result of interference form areas in which the intensity of the ultrasonic radiation is particularly high.

In the ultrasound examination of bony structures having a layer of tissue located in front of them, there are often interferences due to reflection of the ultrasonic radiation at other structures embedded in the layer of tissue, which, as a rule, are considerably smaller than the bony structure whose position and spacing from the outer side of the layer of tissue is to be determined. In order to reduce or completely eliminate the effects of these interferences, provision may be made in accordance with a preferred embodiment of the invention for the position of the transmitters and receivers to be changed in order to thereby direct the ultrasonic radiation on different paths through the layer of tissue towards the structure, and for the signals obtained from the receivers to be averaged over several different paths in order to thereby separate reflections that are produced not by the structure but at interfering structures from reflections from the structure. Owing to the statistical distribution of the individual interfering structures, a different pattern results for each path of the ultrasonic radiation, while the reflection at the structure to be examined is maintained in all cases, and can, therefore, be filtered out in relation to the interference signals during the averaging over several measurements.

For example, the spacings l₁ and/or l₂ of transmitters and receivers can be changed and hence the angle of the ultrasonic radiating direction.

In another method, transmitters and receivers are laterally displaced with the spacing l₁ and l₂, respectively, remaining constant in each case. Since the bony structure, which is to be examined, has, as a rule, a certain extent, the ultrasonic radiation can in this way be guided with a high degree of precision on different paths so that the interfering reflections are substantially suppressed during the averaging.

A further object underlying the invention is to further develop an ultrasound system comprising a transmitter for ultrasonic radiation pulses, a receiver for ultrasonic radiation, a measuring device for determining the transit time of the ultrasonic radiation pulses from transmission by the transmitter to reception of the ultrasonic radiation pulses reflected at a structure, and a data processing device, so that a value of the propagation speed of the ultrasonic radiation, which matches the layer of tissue, is determined with this ultrasound system.

This is accomplished in an ultrasound system of the kind described hereinabove, in accordance with the invention, in that the spacing between the transmitter and the receiver is changeable in such a way that at least two spacings l₁ and l₂, respectively, are settable, and the data processing device is programmed so as to calculate the propagation speed of the ultrasonic radiation in a layer of tissue arranged between transmitter and receiver, on the one hand, and the structure, on the other hand, from the spacings l₁ and l₂ and the transit times t₁ and t₂, respectively, of the ultrasonic pulses, which correspond to these spacings l₁ and l₂.

Such an ultrasound system thus independently transmits on two paths of different length ultrasonic radiation through the layer of tissue to the bony structure, determines the transit time of the ultrasonic pulses and calculates from these two measurements a value of the ultrasound propagation speed, which matches the layer of tissue.

Here it is advantageous for transmitters and receivers to be arranged in a common plane and along a common line for both spacings l₁ and l₂. In particular, the centers of the spacings l₁ and l₂ can coincide.

Provision is made in a particularly preferred embodiment for the ultrasound system to comprise a control device which, in order to bring about the focusing, simultaneously activates several adjacent ultrasound transmitters, so that the ultrasonic rays emanating from these ultrasound transmitters are superimposed on one another and as a result of interference form areas in which the intensity of the ultrasonic radiation is particularly high.

Further provision may be made for the ultrasound system to comprise a control device which changes the position of the transmitters and receivers in order to thereby direct the ultrasonic radiation on different paths through the layer of tissue towards the structure, and for the data processing device to average the signals obtained from the receivers over several different paths in order to thereby separate reflections that are produced not by the structure but at interfering structures from reflections at the structure.

For example, the control device can change the spacings l₁ and/or l₂ between transmitters and receivers and hence the angle of the ultrasonic radiating direction.

In another embodiment, the control device displaces transmitters and receivers laterally with the spacing l₁ and l₂ remaining constant in each case, so that the ultrasonic radiation travels on different paths extending parallel to one another through the layer of tissue.

Provision is made in a particularly preferred embodiment for the ultrasound system to comprise an array of several adjacent transmitters. By activating different transmitters, the spacing from one or possibly also several transmitters can be varied.

It is particularly advantageous for the control device to simultaneously activate several adjacent transmitters of the array. This results in a superimposition of the ultrasonic radiation transmitted by the several transmitters, which leads to a focusing and to an emission of the ultrasonic radiation in a certain direction.

Further provision may be made for the ultrasound system to comprise an array of several adjacent receivers which upon receiving ultrasonic radiation transmit an electric signal to the measuring device.

The transmitters and the receivers may be arranged on a single ultrasound head in a single array, for example, in an array along a straight line or a bent line.

The following description of preferred embodiments of the invention serves in conjunction with the drawings for further explanation.

### BRIEF DESCRIPTION OF THE DRAWINGS

FIG. 1 shows a patient with an ultrasound head placed on the patient, and an ultrasound system for determining the speed of propagation of the ultrasonic radiation in a layer of tissue covering a bony structure;

FIG. 2 shows a diagrammatic view of an array of several ultrasound transmitters and ultrasound receivers with different spacings l₁ and l₂ between transmitters and receivers;

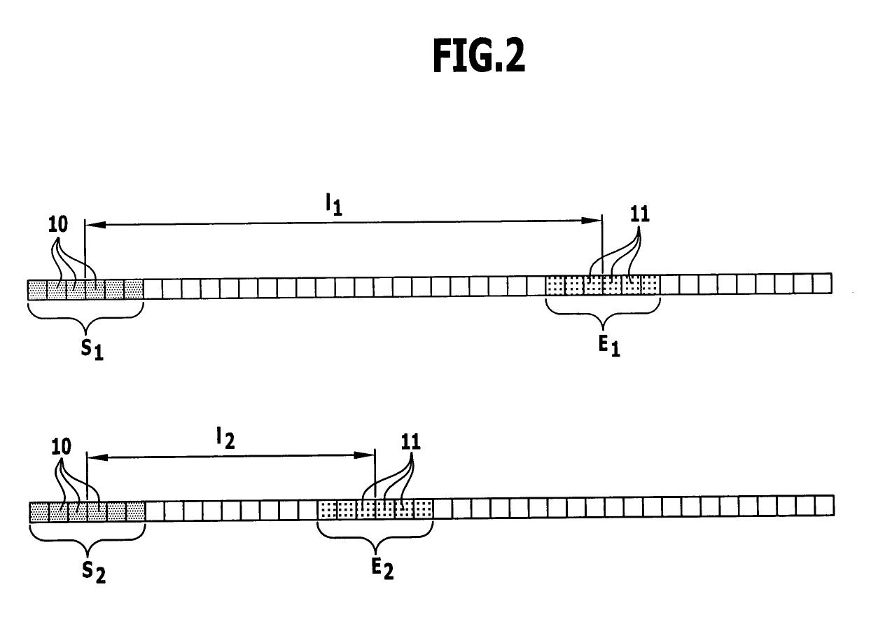

FIG. 3 shows a diagrammatic view of the arrangement of the transmitters and receivers of an ultrasound head on a layer of tissue, and the paths covered by the ultrasonic radiation between a transmitter, a bony structure reflecting the ultrasonic radiation, and a receiver; and

FIG. 4 shows a view similar to FIG. 3 with two different arrangements of transmitters and receivers having the same spacing l₁.

### DETAILED DESCRIPTION OF THE INVENTION

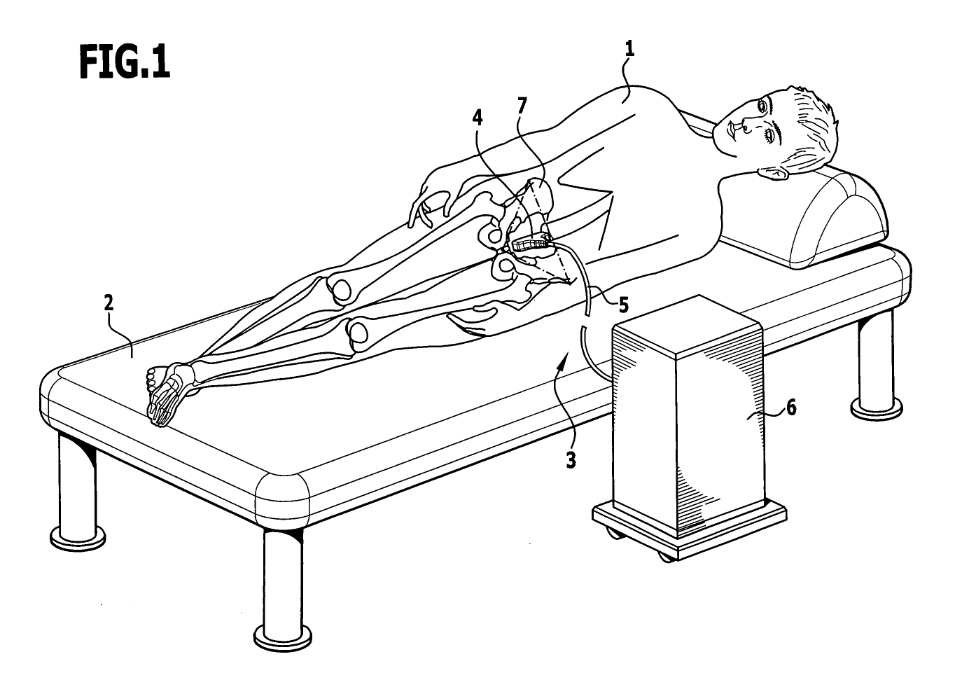

FIG. 1 shows a patient 1 on an operating table 2, who is being examined with the aid of an ultrasound system 3. The ultrasound system comprises an ultrasound head 4, which is connected by a line 5 to a measuring and data processing device 6.

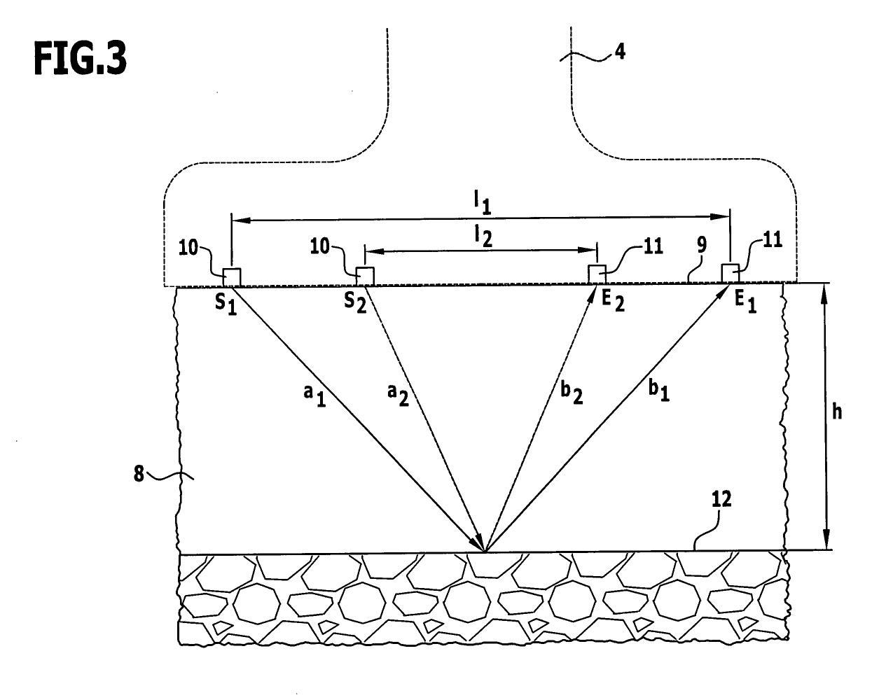

To examine the patient, the ultrasound head 4 is placed on the body surface, for example, at a certain point of the pelvic bone 7, which is covered in this area by a layer of tissue 8. For examination of the patient, ultrasonic radiation is directed from the ultrasound head 4 through this layer of tissue 8 onto the pelvic bone 7. This pelvic bone 7 forms a structure whose spacing from the outer side 9 of the layer of tissue 8 is to be determined (FIG. 3). Hereinbelow the interface between the layer of tissue 8, on the one hand, and the pelvic bone 7, on the other hand, is referred to as bony structure or more simply as structure in contrast to the layer of soft tissue arranged between the ultrasound head 4 and the structure and referred to hereinbelow as layer of tissue 8.

Arranged in the ultrasound head 4 along a preferably straight line immediately adjacent to one another are quite a large number of discrete transmitters 10 and discrete receivers 11. The transmitters 10 can be individually activated by a control device arranged in the measuring and data processing device 6, so that they then transmit an ultrasonic pulse. The receivers 11 can receive ultrasonic radiation impinging on them and generate corresponding electrical signals which are sent through the line 5 to the measuring and data processing device 6.

FIG. 3 shows diagrammatically an ultrasound head 4 placed on the outer side 9 of a layer of tissue 8 with an array-like arrangement of transmitters and receivers, but of the transmitters and receivers of the array only a first transmitter S₁ and a second transmitter S₂, and of the receivers only a first receiver E₁ and a second receiver E₂ are shown, the remaining transmitters and receivers being omitted for reasons of clarity.

The thickness of the layer of tissue 8 is h, and the layer of tissue 8 borders directly on the bony structure 12 at which ultrasonic radiation passing through the layer of tissue is reflected.

To determine the speed of propagation of the ultrasound in the layer of tissue 8, the control device first activates only one transmitter S₁, which directs ultrasonic radiation towards the structure 12. This ultrasonic radiation is reflected at the structure 12, the angle of incidence and the angle of reflection being identical. The path of the ultrasonic radiation between the transmitter S₁ and the structure 12 is designated a1, and the path of the ultrasonic radiation between the structure 12 and a certain receiver E₁, at a certain spacing l₁ from the transmitter S₁, is designated b1. Owing to the identity of the angles of incidence and reflection, the spacing l₁ and the paths a1 and b1 form an equilateral triangle whose center lies immediately above the reflection point on the structure 12.

In an analog manner, for a different transmitter S₂ and a different receiver E₂, at a spacing of l₂ from each other, the path of the ultrasonic radiation is designated a2 and b2, respectively, in FIG. 3. Here, too, the spacing l₂ and the paths a2 and b2 form an equilateral triangle, which in the embodiment of FIG. 3 is so arranged that the tip of this equilateral triangle coincides with the tip of the equilateral triangle formed by the spacing l₁ and the paths a1 and b1. Hence the centers of the spacings l₁ and l₂ lie over each other and over the reflection point of the structure 12.

The measuring device 6 measures the transit time of the ultrasonic pulses between transmission by the transmitters S₁ and S₂ and reception of the reflected radiation by the receivers E₁ and E₂, respectively. Owing to the different lengths of the transit paths, different transit times t₁ and t₂ are measured.

Owing to the identity of the angles of incidence and reflection of the ultrasonic radiation, the paths a1 and b1 are identical to each other, and the paths a2 and b2 are also identical to each other. It is also assumed that the ultrasound propagation speed c is identical throughout the entire layer of tissue 8.

Two equations, namely

4h²=(c·t₁)²+(l₁)²

4h²=(c·t₂)²+(l₂)²

are thus obtained for the initially unknown thickness h of the layer of tissue 8.

In these two equations, the ultrasound propagation speed c and the thickness of the layer of tissue h are unknown, and the remaining quantities are obtained from the measurement. Since two equations are available for the unknown thickness h, the quantity h can be eliminated, which results in the following equation for the ultrasound propagation speed c:

$$c = \sqrt{\frac{{l}_{1}^{2} - {l}_{2}^{2}}{{t}_{1}^{2} - {t}_{2}^{2}}}$$

where l₁ is the spacing between the transmitter S₁ and the receiver E₁, l₂ the spacing between the transmitter S₂ and the receiver E₂, t₁ the transit time of the ultrasonic radiation between the transmitter S₁ and the receiver E₁, and t₂ the transit time of the ultrasonic radiation between the transmitter S₂ and the receiver E₂.

This calculation is made by the data processing device and, therefore, produces a value for the ultrasound propagation speed c in the layer of tissue 8. Although this value is a value which is averaged over the entire layer of tissue, it is a measurement value which matches the nature of the layer of tissue and indicates the actual speed of propagation of the ultrasound far better than a standard value, as was discussed at the outset.

Using this value c, determined by measurement, for the ultrasound propagation speed, the following ultrasound examination can now be carried out in the usual way, and, therefore, the quantity h can also be determined, i.e., the spacing between the bony structure 12 and the outer side or surface of the layer of tissue 8.

In the illustration of FIG. 3, it is assumed that ultrasonic radiation is always only transmitted from a single transmitter S₁ or S₂. In a preferred embodiment, the control device does, however, simultaneously activate several transmitters, which lie immediately adjacent to one another as a group of transmitters, for example, four or five adjacent transmitters, which jointly transmit ultrasonic radiation. With the ultrasonic radiation transmitted from the individual transmitters, this group of transmitters thereby generates an interference pattern, which generates maxima and minima of the ultrasound intensity in a certain direction and at a certain spacing from the transmitters. A focusing on a certain area located in a certain direction and at a certain distance from the group of transmitters is thus possible. The radiation emitted by this group of transmitters is reflected at the bony structure 12 and then travels in a way similar to that described hereinabove to a receiver. The spacing of the receiver from the center of the group of transmitters consisting of individual transmitters corresponds to the above-described spacing l₁ or l₂, and, therefore, in this focused version, too, the ultrasound propagation speed can be determined in the same way from the transit times of the ultrasonic pulses between the two groups of transmitters and the two receivers.

In the embodiment of FIG. 3, transmitters and receivers are shown, which are at two different spacings l₁ and l₂. In principle, it is also possible to work with a larger number of different spacings between transmitters and receivers, as a result of which the ultrasonic rays can assume a larger number of different angles and hence also travel on different paths. Consequently, where there are interfering reflectors in the layer of tissue, the ultrasonic radiation travelling on the different paths will not all be influenced in the same way by these interfering reflectors, as the interfering reflectors are, as a rule, relatively small, whereas the structure 12 to be examined extends over a large area. Owing to the different angles, the ultrasonic rays are, therefore, all reflected by the structure 12, but only some by the interfering reflectors, and, more particularly, in a different way. The influences of the interfering reflectors can, therefore, be reduced or completely eliminated by an averaging, whereas the signals based on the reflection at the structure 12 are maintained in all cases.

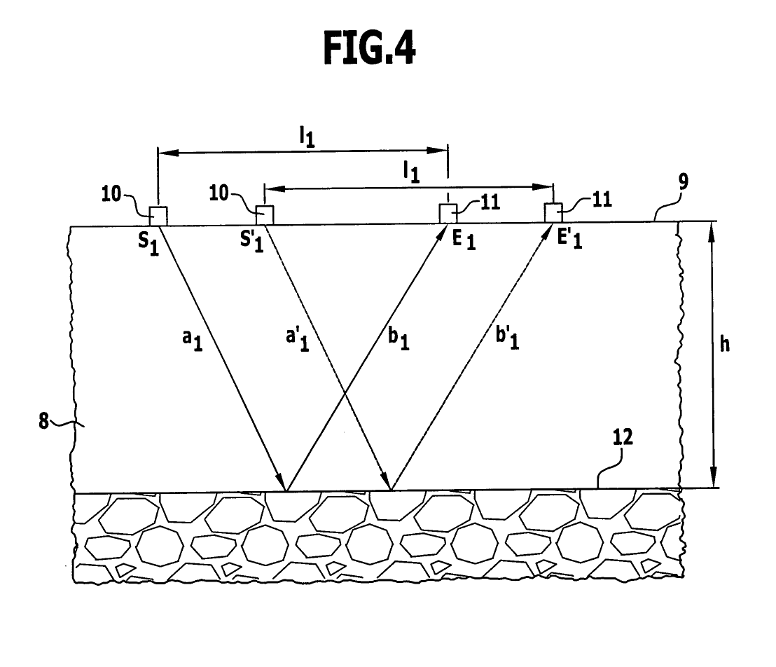

Different paths for the ultrasonic radiation can also be achieved by the ultrasound head being displaced slightly along the layer of tissue with the spacing between transmitter and receiver remaining the same. The paths of the ultrasonic radiation are thereby displaced parallel to themselves and also impinge on different interfering reflectors, whereas all rays continue to impinge on the bony structure 12 which, as a rule, has a larger extent and will, therefore, reflect all ultrasonic rays displaced parallel to themselves. This is illustrated in the example of transmitters S₁ and receivers E₁ in FIG. 4, where different paths are achieved for the ultrasonic radiation by lateral displacement of both the transmitter S₁ and the receiver E₁ with the spacing l₁ remaining the same. The illustration in FIG. 4 does not show the paths of the ultrasonic radiation which, with a different spacing between transmitter and receiver, are additionally obtained, in order to determine the ultrasound propagation speed in the layer of tissue. In this measurement with a different spacing between transmitter and receiver, the procedure is similar to that for the transmitter S₁ and the receiver E₁ in FIG. 4, and, here, too, a different path is prescribed for the ultrasonic radiation by displacing the transmitter and the receiver.

In particular, when a focusing of the radiation is carried out, the measurement can be improved by the thickness h of the layer of tissue being determined after a first measurement in the described manner and by this thus determined thickness being used to focus the ultrasonic radiation at the spacing h from the surface of the layer of tissue 8. The focusing then takes place in the area immediately in front of the bony structure 12, and the overall accuracy and also the sensitivity of the measurement can thereby be successively improved.

1. Method for determining the propagation speed of ultrasonic radiation in a layer of tissue located in front of a structure reflecting ultrasonic radiation, wherein ultrasonic radiation is transmitted from a transmitter arranged immediately outside the layer of tissue through the layer of tissue onto the structure, and the ultrasonic radiation reflected at the structure is received with a receiver arranged immediately outside the layer of tissue, wherein the ultrasonic radiation is transmitted in the form of ultrasonic pulses, and the transit times t₁ and t₂ of the ultrasonic radiation pulses between transmission and reception are determined for at least two different spacings l₁ and l₂ between transmitter and receiver, and the propagation speed of the ultrasonic radiation in the layer of tissue is calculated from the two spacings l₁ and l₂ between transmitter and receiver and from the two transit times t₁ and t₂.

2. Method in accordance with claim 1, wherein transmitter and receiver are arranged in a common plane and along a common line for both spacings l₁ and l₂.

3. Method in accordance with claim 1, wherein transmitter and receiver are so arranged that the centers of their spacing l₁ and l₂, respectively, coincide.

4. Method in accordance with claim 1, wherein the ultrasonic radiation emanating from the transmitter is focused in the area of the structure.

5. Method in accordance with claim 4, wherein the focusing is brought about by several adjacent ultrasound transmitters being simultaneously activated so that the ultrasonic rays emanating from these ultrasound transmitters are superimposed on one another and as a result of interference form areas in which the intensity of the ultrasonic radiation is particularly high.

6. Method in accordance with claim 1, wherein the position of the transmitters and receivers is changed in order to thereby direct the ultrasonic radiation on different paths through the layer of tissue towards the structure, and the signals obtained from the receivers are averaged over several different paths in order to thereby separate reflections that are produced not by the structure but at interfering structures from reflections from the structure.

7. Method in accordance with claim 6, wherein the spacings l₁ and/or l₂ between transmitters and receivers are changed and hence the angle of the radiating direction of the ultrasonic radiation.

8. Method in accordance with claim 6, wherein transmitters and receivers are laterally displaced with the spacing l₁ and l₂, respectively, remaining constant in each case.

9. Ultrasound system comprising a transmitter for ultrasonic radiation pulses, a receiver for ultrasonic radiation, a measuring device for determining the transit time of the ultrasonic radiation pulses from transmission by the transmitter to reception of the ultrasonic radiation pulses reflected at a structure, and a data processing device, wherein the spacing between the transmitter and the receiver is changeable in such a way that at least two spacings l₁ and l₂, respectively, are settable, and the data processing device is programmed so as to calculate the propagation speed c of the ultrasonic radiation in a layer of tissue arranged between transmitter and receiver, on the one hand, and the structure, on the other hand, from the spacings l₁, l₂ and the transit times t₁ and t₂, respectively, of the ultrasonic pulses, which correspond to these spacings l₁, l₂.

10. Ultrasound system in accordance with claim 9, wherein transmitter and receiver are arranged in a common plane and along a common line for both spacings l₁, l₂.

11. Ultrasound system in accordance with claim 9, wherein centers of the spacings l₁, l₂ coincide.

12. Ultrasound system in accordance with claim 9, comprising a control device which, in order to bring about the focusing, simultaneously activates several adjacent ultrasound transmitters, so that the ultrasonic rays emanating from these ultrasound transmitters are superimposed on one another and as a result of interference form areas in which the intensity of the ultrasonic radiation is particularly high.

13. Ultrasound system in accordance with claim 9, comprising a control device which changes the position of the transmitters and receivers in order to thereby direct the ultrasonic radiation on different paths through the layer of tissue towards the structure, wherein the data processing device averages the signals obtained from the receivers over several different paths in order to thereby separate reflections that are produced not by the structure but at interfering structures from reflections at the structure.

14. Ultrasound system in accordance with claim 13, wherein the control device changes the spacings l₁ and/or l₂ between transmitters and receivers and hence the angle of the radiating direction of the ultrasonic radiation.

15. Ultrasound system in accordance with claim 13, wherein the control device laterally displaces transmitters and receivers with the spacing l₁, l₂ remaining constant in each case.

16. Ultrasound system in accordance with claim 9, comprising an array of several adjacent transmitters.

17. Ultrasound system in accordance with claim 16, comprising a control device which simultaneously activates one or more adjacent transmitters of the array.

18. Ultrasound system in accordance with claim 9, comprising an array of several adjacent receivers which upon receiving ultrasonic radiation transmit an electric signal to the measuring device.
